(19) United States
(12) Patent Application Publication
(10) Pub. No.: US 2004/0176747 A1
(43) Pub. Date: Sep. 9, 2004

(54) Catheters

(76) Inventor: Roger Charles Leslie Feneley, Bristol (GB)

Correspondence Address: BLANK ROME LLP, 600 NEW HAMPSHIRE AVENUE, N.W., WASHINGTON, DC, 20037, US

(21) Appl. No.: 10/761,373

(22) Filed: Jan. 22, 2004

Related U.S. Application Data
(63) Continuation of application No. 09/831,945, filed on Jul. 2, 2001, now abandoned.

(30) Foreign Application Priority Data
Nov. 19, 1998 (GB) GB9825253.9

(51) Int. Cl. A61M 27/00

(52) U.S. Cl. 604/544

(57) ABSTRACT

The invention provide suprapubic urethral catheters which preferably consist of a tube having a suprapubic end portion (1) which in use will be located suprapubically in the user, a bladder segment (2) into which in use urine can enter and will be located within the bladder of the user, and a urethral segment (3) distally of the suprapubic end portion which in use will be located within the urethra of the user, the urethral segment (3) including a distensible segment (5) which distends outwardly under pressure within the bladder of the user and is thereby urged into sealing engagement with the interior of the urethra of the user.

This invention relates to catheters for drainage of the urinary bladder.

Urinary incontinence is becoming a major medical, social and economic problem owing to the increasingly aged population. The demographic trends in society indicate that many people are living longer, those over 80 being the fastest growing section of the population, and the prevalence of urinary incontinence increases with age. Long term catheterisation of the bladder is instituted when all other methods of managing urinary incontinence have been excluded. The high morbidity is unacceptable, yet it remains the mainstay of management for many elderly people, with 2% requiring such catheterisation by the age of 85 years.

The self-retaining balloon catheter introduced by Dr Foley in 1935 has been the standard product for over 60 years. However, a major problem with these catheters is that they tend to block and to allow urine to pass around the outside of the catheter, and patients with these catheters require close nursing supervision

According to the present invention there is provided a suprapubic urethral catheter.

Catheters in accordance with the present invention preferably consist of a tube having a suprapubic end portion which in use will be located suprapubically in the user, a bladder segment into which in use urine can enter and will be located within the bladder of the user, and a urethral segment distally of the suprapubic end portion which in use will be located within the urethra of the user, the urethral segment including a distensible portion which distends outwardly under pressure from within the bladder of the user and is thereby urged into sealing engagement with the interior of the urethra of the user.

An embodiment of catheter in accordance with the present invention will now be described with reference to the accompanying diagrammatic drawings in which:—

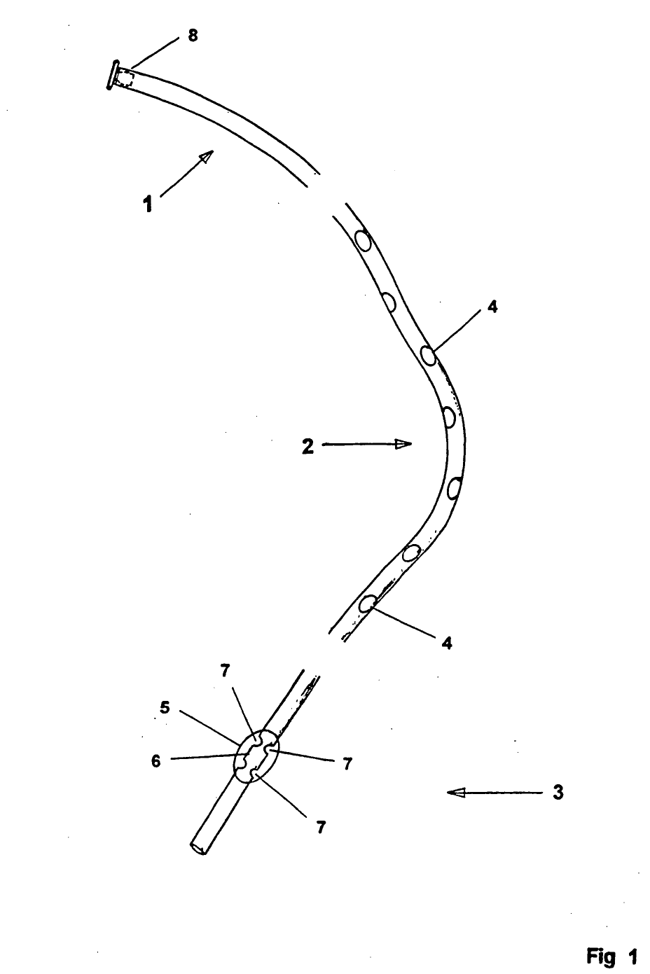

FIG. 1 is a part cut away view of the embodiment;

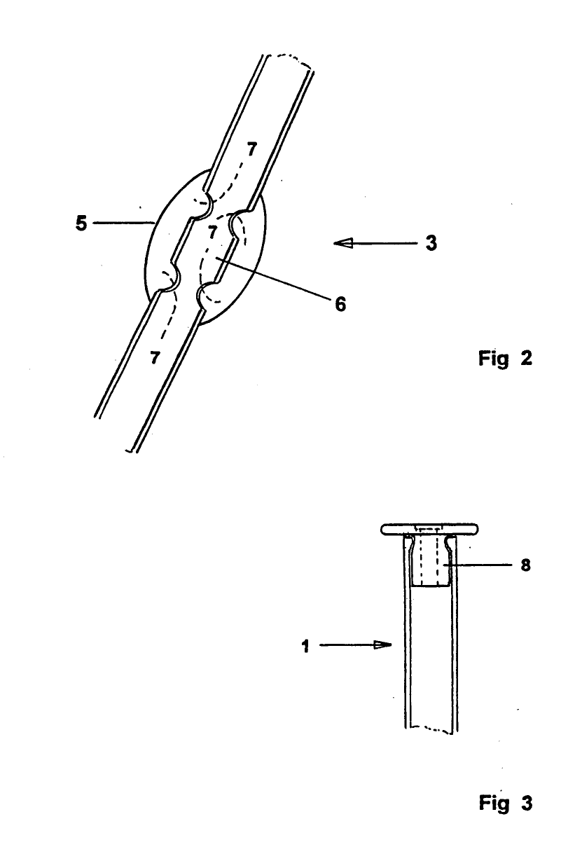

FIG. 2 is a view to an enlarged scale of one end portion of the embodiment; and

FIG. 3 is a view to an enlarged scale of the opposite end portion of the embodiment from that of FIG. 2.

Referring to FIG. 1, the catheter consists of a flexible plastics tube having a suprapubic end portion 1, a bladder draining segment 2, and at the opposite end a urethral end portion 3.

The bladder draining segment 2 has a plurality of holes 4 which in use allow urine in the bladder to drain into the interior of the tube, urine then flowing through the tube towards the urethral end portion 3 under gravity.

The urethral end portion 3 has a distensible segment 5 which surrounds and is integral with a section 6 of the end portion 3, the section 6 having a plurality of apertures 7 through which urine can pass from the interior of the tube into the dispensible segment 5 and inflate it like a balloon. The distensible segment 5 will usually extend for from 2 to 2.5 cm along the length of the section 6 in order to provide good sealing engagement of the external surface of the distensible segment 5 with the internal surface of the urethra when the distensible segment 5 is inflated by the pressure of urine within the bladder of the user.

When located within a user, the suprapubic end portion 1 is positioned through the abdominal wall of the user, the bladder draining segment 2 is positioned within the bladder, and the urethral end portion 3 is positioned within the urethra with the distensible segment 5 located so that when inflated it forms a substantially urine tight seal with the internal surface of the urethra.

The upper end of the suprapubic portion 1 will in general be closed by a non-return valve 8 and be held in place by a flange (not shown) which retains the tip of the catheter on the surface of the body of the user. The non-return valve not only serves to prevent urine from escaping from the bladder, it can facilitate access to the interior of the catheter from its suprapubic end, for example by allowing a connector to be passed through the valve to drain or irrigate the bladder, or for cleaning the interior of the catheter using a fine brush or “pipe cleaner”.

The urethral end portion 3 will in general be provided with closure means (not shown) which prevents urine entering the drainage holes 4 from continuously pouring out of the lower end of the catheter. Such closure means serves the purpose not only of controlling the discharge of urine, it also results in urine collecting within the urethral end portion 3 and then entering the distensible segment 5, with the result that the distensible segment 5 becomes inflated. As a result, the user's bladder can fill with urine without leakage between the distensible segment 5 and the urethra, and indeed as the pressure of urine within the bladder increases, it generally serves to improve the seal there between.

In some patients it may not be necessary to provide the urethral portion with the distensible segment, for example if continual drainage into a collection bag is required. In some patients the catheter may terminate above the urethral sphincter, thus allowing the patient to control the urinary output voluntarily.

Control of urinary discharge can then be effected by continuous or intermittent drainage which can be from either end of the catheter, drainage being controlled, for example, using a known type of valve. Thus a simple on/off valve of a type-which is currently commercially available could be used, or a remotely controlled valve could be used which is operable by a control button. Alternatively, where drainage of the bladder is to be effected automatically, a valve can be used which allows urine to be drained from the bladder at regular time intervals.

Although the illustrated embodiment uses a distensible segment 5 into which urine can pass to provide a seal between the external surface of the catheter and the internal surface of the urethra, other distensible means can be used for the purpose, such means being urged into increased sealing contact with the interior of the urethra when the pressure of urine within the bladder increases. As will also be appreciated, the distensible segment 5 can be omitted, for example for patients capable of voluntarily controlling urinary output.

Catheters in accordance with the present invention should obviously be flexible, and they are preferably made of a flexible plastics material, for example polyurethane, silicone rubber or polyvinyl chloride.

1. A suprapubic urethral catheter.

2. A catheter according to claim 1, having a urethral segment with a distensible segment which distends outwardly under pressure from within the bladder of the user and is thereby urged into sealing engagement with the interior of the urethra of the user as pressure within the bladder increases.

3. A catheter according to claim 2, having a suprapubic end portion which in use will be located suprapubically in the user.

4. A catheter according to claim 2 or claim 3, having a bladder segment into which urine can enter when the catheter is located within the bladder of the user.

5. A catheter according to any of claims 2 to 4, wherein the suprapubic end portion includes a non-return valve for preventing the escape of urine from said end portion.

6. A catheter according to claim 4, wherein the bladder segment has a plurality of holes therein for allowing urine to enter it.

7. A catheter according to any of claims 2 to 6, wherein the distensible portion comprises a balloon.

8. A catheter according to any of the preceding claims, including control means for controlling the outflow of urine therefrom.

9. A catheter according to claim 8, wherein the control means comprises a manually operable tap or remotely controllable valve.

10. A suprapubic urethral catheter substantially as herein described with reference to the accompanying drawings.
